(19) United States
(12) Patent Application Publication
(10) Pub. No.: US 2012/0276526 A1
(43) Pub. Date: Nov. 1, 2012

(54) METHOD AND SYSTEM FOR MONITORING AND RECORDING VIRAL INFECTION PROCESS AND SCREENING FOR AGENTS THAT INHIBIT VIRUS INFECTION

(75) Inventors: Chih-Wei Wu, Keelung City (TW); Chang-Jer Wu, Taipei City (TW); Shih-Hao Huang, Kaohsiung City (TW); Sheng-Ping Chang, New Taipei City (TW)

(73) Assignee: National Taiwan Ocean University, Keelung City (TW)

(21) Appl. No.: 13/547,323

(22) Filed: Jul. 12, 2012

Related U.S. Application Data
(63) Continuation-in-part of application No. 13/185,996, filed on Jul. 19, 2011.

(30) Foreign Application Priority Data
Jan. 20, 2011 (TW) 100102179

(51) Int. Cl. C12Q 1/70; C12M 1/34

(52) U.S. Cl. 435/5; 435/287.1; 435/288.7

(57) ABSTRACT

The present invention relates to a method for monitoring and recording a viral infection process, which is characterized by providing a microcantilever detection device, which comprises a microcantilever comprising a contact area having an macromolecular material attached thereon; loading host cells to the contact area to allow the host cells to be attached to the macromolecular material; loading virus to the contact area to make the virus to contact the host cells attached thereto whereby a deflection level of the microcantilever is produced; and recording the deflection level in a time course manner so as to obtain a deflection curve that can be used as a basis for monitoring and recording the viral infection process. The method of the invention can also be used for screen for an agent that inhibits virus infection.

### CROSS-REFERENCE TO RELATED APPLICATION PARAGRAPH

This application is a continued-in-part application of patent application Ser. No. 13/185,996, filed on Jul. 19, 2011, which claims the benefit of Taiwan Patent Application No. 100102179 filed on Jan. 20, 2011, the content of which is hereby incorporated by reference in their entirety.

### TECHNOLOGY FIELD

The present invention relates to a method and system for monitoring and recording a viral infection process and screening for agents that inhibit virus infection, such as vaccines, drugs and health food.

### BACKGROUND OF THE INVENTION

In general, a viral infection process in host cells comprises three phases: (1) the penetration phase: targeting and entering the host cells; (2) the replication phase: replicating nucleic acids and proteins needed for the virus to live; and (3) the transmission phase: leaving the infected cell and further infecting other cells. A thorough understanding of the viral infection process not only provides a basis for developing anti-virus drugs or vaccines, but also is helpful for providing accurate timing for administering medicine, so as to treat diseases effectively. However, most of the current virus detection technologies, including reverse transcriptase-polymerase chain reaction (RT-PCR), virus isolation, enzyme-linked immunosorbent assay (ELISA), polymerase chain reaction (PCR) and the like, as well as microcantilever biological sensor technology that is being developed recently, are to detect the presence of a virus based on specific binding interaction between certain molecules, such as specific interaction between primers, probe or antibodies and antigens, but none of those is used to monitor and record a complete viral infection process in host cells.

In addition, current methods for screening for a candidate agent for inhibiting virus infection is mainly based on cell or animal experiments, which are time-consuming, expensive and inefficient.

### BRIEF SUMMARY OF THE INVENTION

The present invention relates to a detection technology, which can be used for monitoring and recording a viral infection process in host cells, characterized by providing a microcantilever detection device, which comprises a microcantilever comprising a contact area having an macromolecular material attached thereon; loading host cells to the contact area to allow the host cells to be attached to the macromolecular material; loading a sample comprising a virus to the contact area to make the virus to contact and infect the host cells attached thereto whereby a deflection level of the microcantilever is produced; and measuring the deflection level in a time course manner so as to obtain a deflection curve (also called the control deflection curve) that can used as a basis for determining the viral infection process.

The detection technology of the present invention can also be used to preliminary screen for an agent that inhibits virus infection. To evaluate if a test agent has potential to inhibit virus infection in host cells, a separate detection using microcantilever detection device of the invention is independently conducted in the same manner, except that the test agent is added to the sample that is to be loaded to the contact area of the microcantilever, and the deflection curve thus obtained (with the test agent) is compared with the control deflection curve (without the test agent); if the deflection curve thus obtained (with the agent) exhibits a less steeper slope (more steady) than the deflection control curve (without the agent), that means that the test agent is a candidate for inhibiting virus infection in the host cells.

Accordingly, in one aspect, the present invention provides a method for monitoring and recording a viral infection process, comprising:

(a) providing a microcantilever detection device, which comprises a microcantilever comprising a contact area having a macromolecular material attached thereon; wherein the macromolecular material is hydrophilic and biocompatible;

(b) loading host cells to the contact area to allow the host cells to be attached to the macromolecular material, and washing the contact area to remove unattached cells;

(c) loading a sample containing a virus capable of infecting the host cells to the contact area to allow the virus to contact and infect the host cells attached thereto, and washing the contact area to remove free virus that does not infect the host cells, whereby the microcantilever causes a deflection level;

(d) measuring the deflection level of the microcantilever in a time course manner during a period of time so as to give a deflection curve; and

(e) monitoring and recording an infection process of the virus in the host cells based on the deflection curve, in which when a continued increase of the deflection level appears, it indicates that the virus replicates in the host cells, and when the deflection level achieves a maximum value, it indicates that the virus completes replication in the host cells and starts to leave the host cells.

In another aspect, the present invention provides a method of evaluating if a test agent inhibits virus infection, comprising:

(a) conducting a first detection as a control, which comprises

(i) providing a microcantilever detection device, which comprises a microcantilever comprising a contact area having a macromolecular material with host cells attached thereto, wherein the macromolecular material is hydrophilic and biocompatible;

(ii) loading a sample containing a virus that is capable of infecting the host cells to the contact area to allow the virus to contact and infect the host cells, and washing the contact area to remove free virus that does not infect the host cells, whereby the microcantilever causes a first deflection level, which continuously increases as the virus replicates in the host cells over time; and

(iii) measuring the first deflection level in a time course manner during a period of time so as to give a first deflection curve, which has a first slope representing the increase of the first deflection level caused by replication of the virus in the host cells,

(b) conducting a second detection in the same manner as in the first detection, except that the test agent is added to the sample to be loaded to the contact area, so as to give a second deflection curve, which has a second slope corresponding to the first slope in the first deflection curve;
(c) comparing the second slope in the second deflection curve with the first slope in the first deflection curve, wherein the second slope less steeper than the first slope indicates that the test agent is a candidate inhibiting infection of the virus in the cells.

In a further aspect, the present invention provides a system for monitoring and recording an infection process of a virus in host cells, comprising:

(a) a microcantilever detection device, which comprises a microcantilever comprising a contact area having a hydrophilic and biocompatible macromolecular material attached thereon for fixing the host cells, and a signal detecting area; wherein when the host cells are attached to the contact area, and the virus is then loaded to the contact area and contacts and infects the host cells, the microcantilever produces a deflection level that is detectable through the signal detecting area;

(b) a signal detecting device, comprising a signal producing means for producing a detectable signal responsible to the deflection and a signal receiving means for receiving the detectable signal and converting it to an outputting signal; and

(c) a signal processing device for receiving the outputting signal and converting it to a data so as to give a deflection curve in a period of time of measurement, which presents the infection process of the virus in the host cells.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows an embodiment according to the microcantilever detection device of the present invention.

FIG. 2 shows (a) a specific embodiment according to the microcantilever detection device of the present invention (π-shaped microcantilever); and (b) the fabrication process of the microcantilever and PDMS microfluidics in one embodiment of the invention; and (c) use of the gelatin gel as a sacrificial layer to solve the sticking problem between the PDMS microfluidics and the hydrogel microstructure.

FIG. 3 shows a diagram of the detection technology for monitoring and recording the viral infection process in host cells in one embodiment of the present invention, wherein [symbol] means the flow direction, [symbol] means a macromolecular material (e.g. hydrogel), [symbol] means UV light, [symbol] means host cells, [symbol] means a test virus, (a) means that a macromolecular material (e.g. hydrogel) solution is added to the microcantilever; (b) means UV light exposure; (c) means that the macromolecular material (e.g. hydrogel) is cured into a solid, the host cells are loaded to the macromolecular material, and the microcantilever generates a deflection level h1; (d) means that a sample containing the test virus is loaded to the macromolecular material (e.g. hydrogel); (e) means that the test virus is attached to the macromolecular material (e.g. hydrogel), and the microcantilever generates a deflection level h2; (f) means that the virus enters and replicates in the host cells, and the microcantilever generates a deflection level h3; and (g) means that virus leaves the host cells, and the deflection level goes back to h0.

FIG. 4 shows an embodiment according to the microcantilever detection device of the present invention 101, which comprises a microcantilever 2, a hydrogel material 6 attached to the contact area thereof, PDMS microfluidics 14, a silicon wafer substrate 16, a microfluidic inlet 141 and a microfluidic outlet 142.

FIG. 5 shows an embodiment according to the detection system of the present invention, comprising a He—Ne laser source 102, a space filter 104, a pinhole 105, focus lens 106, refractive lens 108, a position sensing detector 110, a microcantilever detection device 101, a first reflected beam 118, a second reflected beam 120, and a signal processing device 112.

FIG. 6 is a photograph showing that the microfluidic system chip was bond to the microcantilever chip as described in Example 1.

FIG. 7 is a diagram showing the relationship between the light signal and the displacement of the microcantilever, wherein the laser beam 118 is focused on the optical detection area of the microcantilever 2, and the beam is reflected to the four-quadrant position sensing detector 110; and the displacement d of the reflected light is measured, from which the deflection ΔZ of the microcantilever detection device is calculated, wherein d means the displacement of the reflected laser beam on the four-quadrant position sensing detector; h means the distance between microcantilever detection device and the four-quadrant position sensing detector; and L means the length of the microcantilever detection device.

FIG. 8 shows a specific example of an optical detection system according to the present invention, including a He—Ne laser source 102, a space filter 104, focus lens 106, refractive lens 108, a position sensing detector 110, a microcantilever detection device 101, a charge-coupled device (CCD) 114, and a sample stage 116.

FIG. 9 shows the deflection curve of the microcantilever in one embodiment of the invention obtained as in Example 3.

FIG. 10 shows the deflection curve of the microcantilever in one embodiment of the invention obtained as in Example 3.

FIG. 11 shows the situations where the host cells were attached to the hydrogel material and infected by the viruses as in Example 3, which were simultaneously observed with a fluorescence microscope of the optical detection system according to the present invention. Scale: 200 μm. The image (a) refers to the control group showing that the cells were mostly alive, (b) refers to the experimental group at 4 hours after virus infection, (c) refers to the experimental group at 7 hours after virus infection, and (d) shows that most of the cells died and detached from the surface of the hydrogel material.

FIG. 12 shows the deflection curve according to the present invention detected in Example 3. The time point (a) 09:55:31 AM is a start point (loading the cells); at (b) 04:33:55 PM, the wash step was conducted; at (c) 07:40:36 PM, the virus was loaded; at (d) 08:37:48 PM, the wash step was conducted; and at (e) 01:16:16 AM, the deflection level reached a maximum value, indicating that the virus completed replication in the host cells and was leaving the host cells (transmission phase).

FIG. 13 (a) shows the deflection curves according to the present invention detected in Example 4.1 (JEV curve, JEV+NS1 curve, and JEV+IgG curve). FIG. 13 (b) shows the fluorescence microscopic images of the cells in the study.

FIG. 14 (a) shows the deflection curves according to the present invention detected in Example 4.2 (DV curve, DV+IgG curve, and DV+NS3 curve). FIG. 14 (b) shows the fluorescence microscopic images of the cells in the study.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The present invention relates to a method and system for monitoring and recording a viral infection process in cells and that for screening for an agent for inhibiting virus infection by using a microcantilever detection technology.

In one aspect, the present invention provides a method for monitoring and recording a viral infection process, comprising:

(a) providing a microcantilever detection device, which comprises a microcantilever comprising a contact area having a macromolecular material attached thereon; wherein the macromolecular material is hydrophilic and biocompatible;

(b) loading host cells to the contact area to allow the host cells to be attached to the macromolecular material, and washing the contact area to remove unattached cells;

(c) loading a sample containing a virus capable of infecting the host cells to the contact area to allow the virus to contact and infect the host cells attached thereto, and washing the contact area to remove free virus that does not infect the host cells, whereby the microcantilever causes a deflection level;

(d) measuring the deflection level of the microcantilever in a time course manner during a period of time so as to give a deflection curve; and

(e) monitoring and recording an infection process of the virus in the host cells based on the deflection curve, in which when a continued increase of the deflection level appears, it indicates that the virus replicates in the host cells, and when the deflection level achieves a maximum value, it indicates that the virus completes replication in the host cells and starts to leave the host cells.

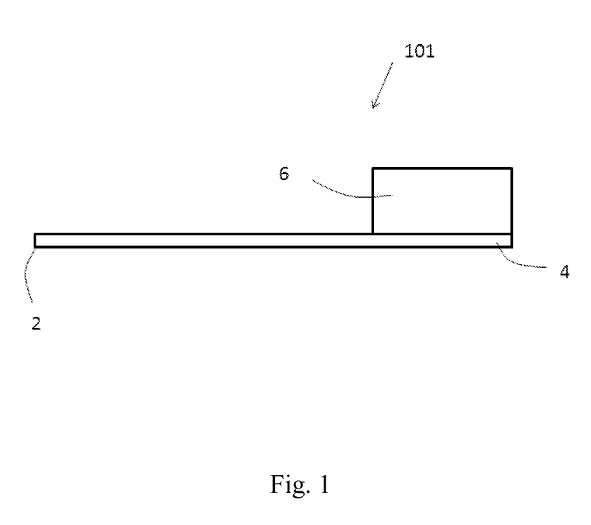

As shown in FIG. 1, in an embodiment according to the present invention, a microcantilever detection device 101 comprises a microcantilever 2, wherein the microcantilever comprises a contact area 4, on which a macromolecular material 6 is attached.

As used herein, the term “macromolecular material” is a hydrophilic and biocompatible macromolecular material, which can be used to fix cells and is non-toxic to the cells. In one embodiment, the macromolecular material is a hydrogel material.

As used herein, the term “hydrogel material” is a water-absorbing gel. Hydrogel material is hydrophilic and biocompatible, which can absorb water to swell while keeping its three dimensional (3D) structure. There are various hydrogel materials that can be used herein, including but are not limited to, polyhydroxyethylmethacrylate (PHEMA) hydrogel, polyethylene glycol diacrylate (PEGDA) hydrogel, gelatin methacrylate (GelMA) hydrogel, alginate hydrogel, alginate hydrogel, chitosan hydrogel and agarose hydrogel and the like. In order to allow a hydrogel material to be fixed on microcantilever, typically, a crosslinking agent and a photoinitiator are added to a hydrogel material solution, to allow the hydrogel material solution to be used in amicroforming process, and the hydrogel material is cured into a solid in the contact area of the microcantilever by using an exposure method.

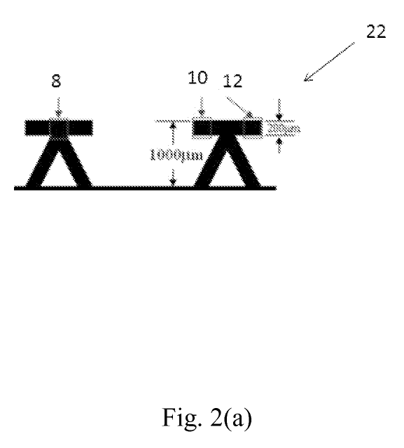

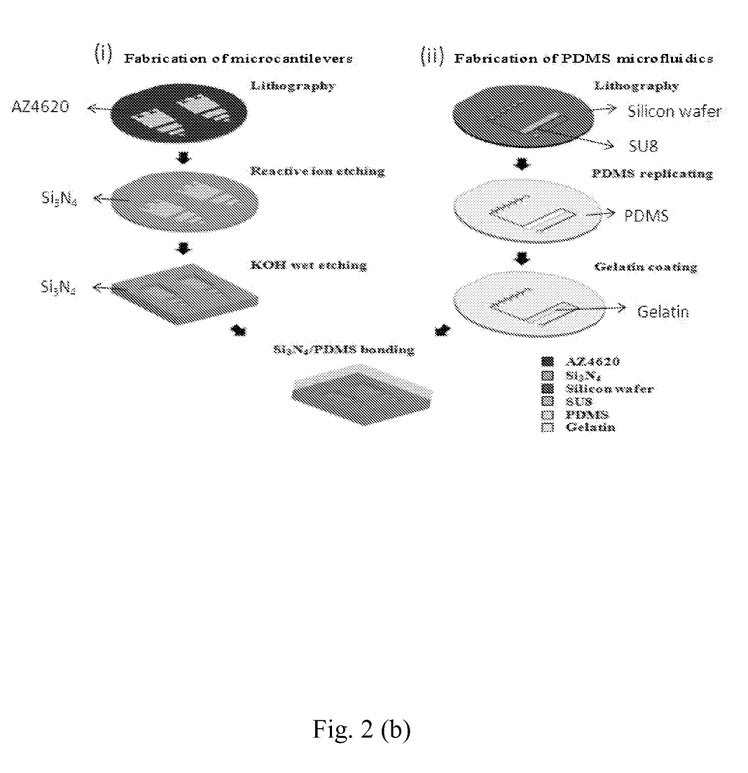

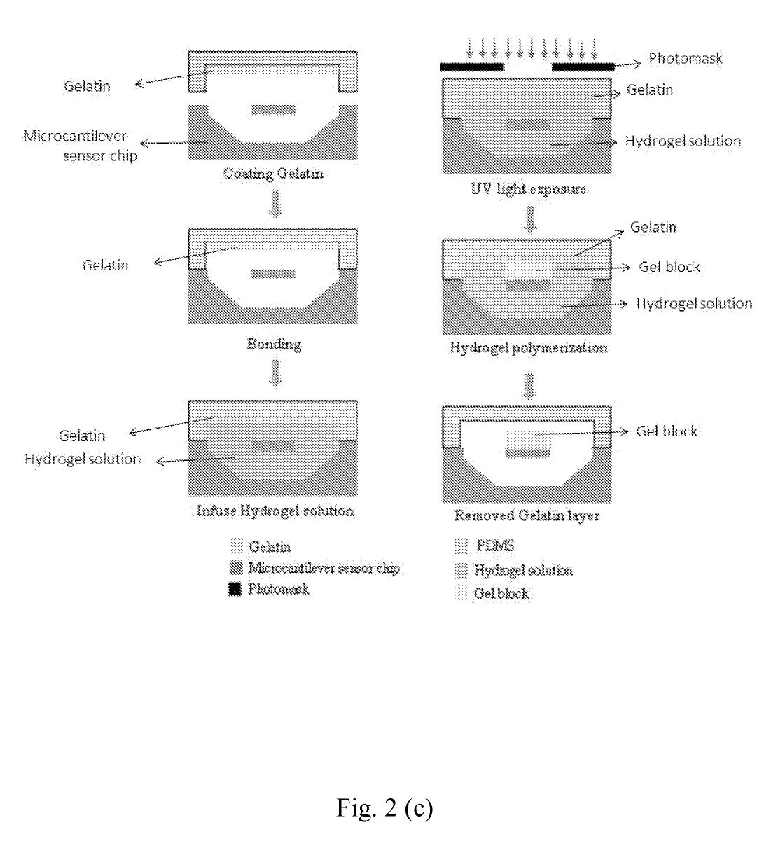

As used herein, the term “microcantilever” may be that used in a microcantilever sensor, for example, generally used in this art, which produces a small deformation in structure with a slight force (e.g. deflection), and a detection with high sensitivity can be conducted based on such small deformation. In one embodiment, the microcantilever is π-shaped. Other shapes of the microcantilever include but are not limited to H-shape, K-shape, Y-shape, X-shape, T-shape, W-shape and M-shape. As shown in FIG. 2 (a), in a certain embodiment, the microcantilever of the present invention is a π-shaped microcantilever 22, having a hydrogel material as the macromolecular material for fixing host cells, wherein the middle part of the π-shaped microcantilever 22 is designed as a hydrogel material exposing area 8, the size of which is about 200 μm×200 μm that is designed based on the line width of the microcantilever; both ends of the π-shaped microcantilever 10, 12 may be used as optical detection areas for the incident and reflected laser beam, to avoid light shining directly on the uneven hydrogel material, or to avoid refraction which may cause consequential difficulty for a position sensing detector to receive a light signal and subsequently inaccuracy of signal measurement, and also prevent cells from damages caused by the high energy laser beam.

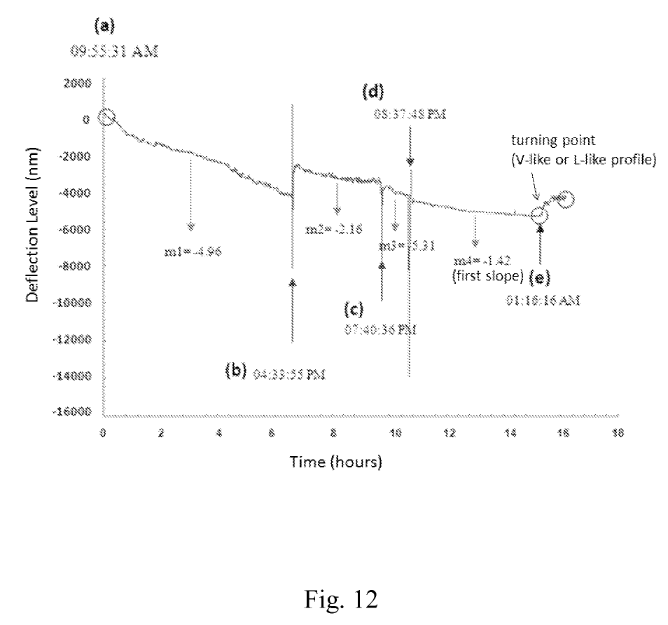

It is demonstrated based on the synchronous observation via fluorescence microscope that, the deflection curve obtained according to the present invention can represent a viral infection process in host cells. As mentioned above, a viral infection process in host cells typically includes a penetration phase, a replication phase and a transmission phase. According to the invention, when an increase of the deflection level is observed in the deflection curve (after a sample comprising the virus is loaded to the contact area of the microcantilever), it indicates that the virus replicates in the host cells, and when the deflection level achieves a maximum value (a turning point) after which the deflection level stops increasing and starts to go towards to an original level prior to loading of the sample (move upwards), it indicates that the virus completes replication in the host cells and starts to leave the host cells (entering the transmission phase). Further, around the turning point, the defection curve forms an “V-like” or “L-like” profile (bouncing back), which is an important marker representing that the replication phase is completed and the virus is leaving the cells. See FIG. 12, for example.

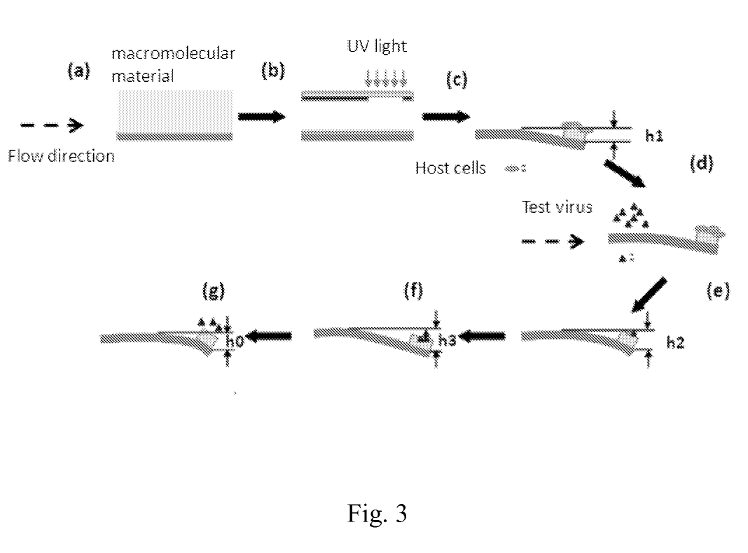

In one embodiment of the present invention, as shown in FIG. 3, a hydrogel material is used as the macromolecular material for fixing host cells (a). The hydrogel material solution is cured to a solid on the microcantilever through UV irradiation (b), and then a sample containing host cells is loaded to the microcantilever (c) so that the host cells are attached to the hydrogel material; at this time, the microcantilever produces a deflection h1. Subsequently, a sample containing a virus is loaded to the microcantilever to allow the virus contacts and infects the host cells (d). During the infection process, the virus contacts the cells (penetration phase), replicates in the cells (replication phase), and leaves the cells (transmission phase), and microcantilever generates deflections h2, h3 and h0 at different phases, wherein h2 represents the penetration phase (e), h3 represents the replication phase (f), and h0 represents the transmission phase (g) that the virus leaves the host cells, wherein h3>h2>h1, and h0<h3; i.e. the deflection level reaches a maximum value (h3) and then tends to go back toward the original level prior to loading of the sample containing the virus (h1), which indicates that the virus completes replication in the cells and starts to leave the host cells so that the deflection level decreases (move upwards).

The deflection level generated by the microcantilever may be detected by a common detection methods known in this art, including but are not limited to, an optical detection approach, an acoustic detection approach, an electric detection approach, or a magnetic detection approach.

In one embodiment, the microcantilever detection device of the present invention may further comprises a microfluidic system, through which a sample containing the test virus is loaded to the contact area of the microcantilever to allow the test virus to contact and infect the host cells fixed thereto.

Typically, the microcantilever detection device of the present invention is fabricated using a four-inch silicon wafer. The fabrication procedures of the microcantilever begins with a low stress silicon nitride layer deposited on a four-inch silicon wafer followed by a photolithographic process using a photoresist, which defines the pattern of the microcantilever, and a reactive ion etching system is then used to selectively etch away the undefined photoresist portion of low stress silicon nitride by SF₆ and O₂ gases. The uncovered portion of the silicon substrate was etched by potassium hydroxide solution to release the microcantilevers. On the other hand, the fabrication procedures of a polydimethylsiloxane (PDMS) microfluidic system typically begin with a silicon wafer coated with a photoresist, to create a mold master, including an inlet, an outlet, several bubble traps and a chamber for cells adhered and cultured on the microcantilever. Following soft baking of the photoresist, the photomask patterns are transferred to the photoresist coated silicon wafer. Post-exposure bake, development, and hard bake of the exposed photoresist patterns are followed by pouring the mixture of PDMS onto the patterns. The master is then replicated with PDMS as shown in FIG. 2 (b). A thin gelatin film used as a sacrificial layer is then deposited onto the bottom of the cell laden chamber of the PDMS replica to create a space for cells to be laden and cultured and to avoid the hydrogel microstructure sticking to the PDMS replica when hydrogel was exposed to UV light from the top side of the PDMS replica (see FIG. 2(b)). A hydrogel material solution is prepared and loaded to the microcantilever through the microfluidic system and cured into a solid on the microcantilever, and subsequently the remaining hydrogel solution is washed away. Finally, the surface modified PDMS cast is bonded onto the silicon wafer containing a microcantilever by using oxygen plasma bonding technology, and the microcantilever detection device of the present invention is accomplished.

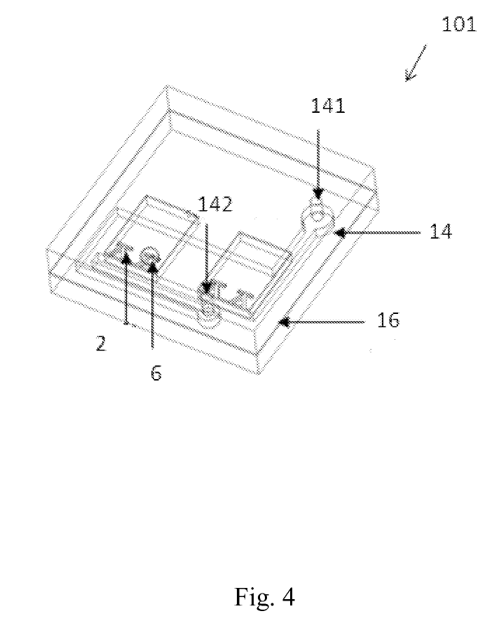

In one embodiment, as shown in FIG. 4, the microcantilever detection device of the present invention 101 comprises a microcantilever 2, a hydrogel material 6 attached on the contact area of the microcantilever, a PDMS microfluidic system 14, a silicon wafer substrate 16, an inlet of the microfluidic system 141 and an outlet of the microfluidic system 142.

In addition, in a further aspect, the present invention provides a system for monitoring and recording an infection process of a virus in host cells, comprising:

(a) a microcantilever detection device, which comprises a microcantilever comprising a contact area having a hydrophilic and biocompatible macromolecular material attached thereon for fixing the host cells, and a signal detecting area; wherein when the host cells are attached to the contact area, and the virus is then loaded to the contact area and contacts and infects the host cells, the microcantilever produces a deflection level that is detectable through the signal detecting area;

(b) a signal detecting device, comprising a signal producing means for producing a detectable signal responsible to the deflection and a signal receiving means for receiving the detectable signal and converting it to an outputting signal; and

(c) a signal processing device for receiving the outputting signal and converting it to a data so as to give a deflection curve in a period of time of measurement, which presents the infection process of the virus in the host cells.

According to the present invention, the signal detecting device may be established based on optical detection approach, an acoustic detection approach, an electric detection approach, or a magnetic detection approach. In one embodiment, the signal detecting device is establish based on an optical detecting approach (i.e. an optical detecting device), which comprises a laser source, a spatial filter, a focusing lens set, refractive lens, a position sensing detector, wherein the laser source provides a beam of laser light that goes through the space filter to form an uniform beam, which then goes through the focusing lens set to form a parallel beam, which further goes through the refractive lens to form a first reflected beam 118, which is subsequently focused on the signal detecting area of said microcantilever detection device and forms a second reflected beam 120, and the position sensing detector receives the second reflected beam and converting it to an electrical outputting signal.

In a certain example, the system of the invention further comprises a charge coupled device for observing whether the first reflected beam is focused on the signal detecting area of said microcantilever detection device. In another example, said charge coupled device may also be used for observing the host cells, for example, to confirm the cell viability and the different phases of the infection process.

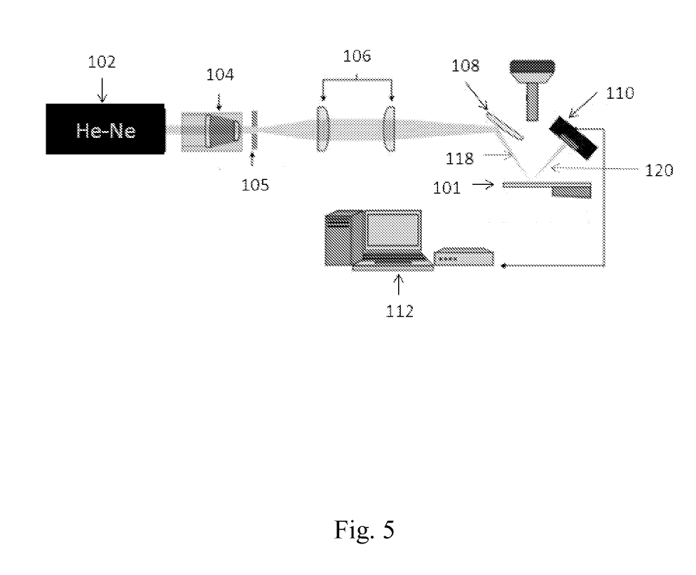

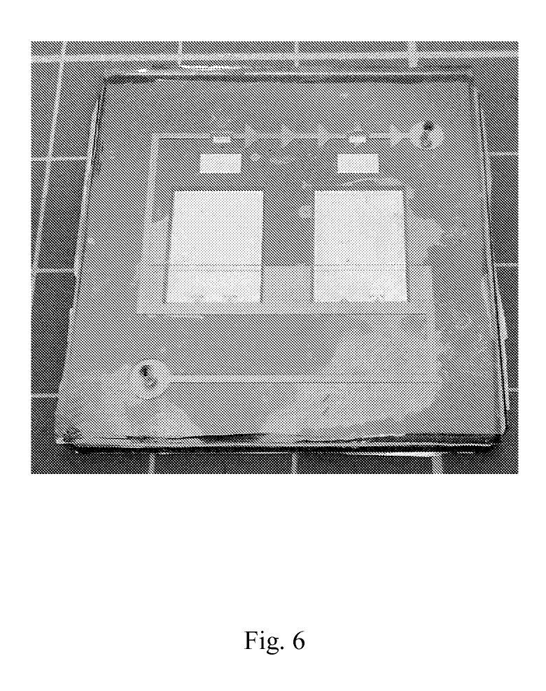

As shown in FIG. 5, in one embodiment, the system of the present invention is an optical detection system, comprising a He−Ne laser source 102, a space filter 104, a pinhole 105, a focusing lens set 106, refractive lens 108, a position sensing detector 110, a microcantilever detection device 101, a first reflected beam 118, a second reflected beam 120, and a signal processing device 112.

In one specific example, the system of the present invention comprises:

(a) a microcantilever detection device, which comprises a microcantilever comprising a contact area having a hydrophilic and biocompatible macromolecular material attached thereon for fixing the host cells, and a signal detecting area; wherein when the host cells are attached to the contact area, and the virus is then loaded to the contact area and contacts and infects the host cells, the microcantilever produces a deflection level that is detectable through the signal detecting area;

(b) an optical detecting device, comprising a laser source, a spatial filter, a focusing lens set, refractive lens, a position sensing detector, wherein the laser source provides a beam of laser light that goes through the space filter to form an uniform beam, which then goes through the focusing lens set to form a parallel beam, which further goes through the refractive lens to form a first reflected beam, which is subsequently focused on the signal detecting area of said microcantilever detection device and forms a second reflected beam, and the position sensing detector receives the second reflected beam and converts it to an electrical outputting signal; and

(c) a signal processing device for receiving the outputting signal and converting it to a data so as to give a deflection curve in a period of time of measurement, which presents the infection process of the virus in the host cells.

The technology of the invention can also be used for screening for an agent for inhibiting virus infection. To accomplish this purpose, a separate detection is conducted according to the invention wherein a sample containing the virus and a test agent to be evaluated is loaded to the contact area of the microcantilever, whereby a separate detection curve is obtained, and if the separate detection curve shows a less steeper slope when comparing with the control detection curve (without adding the test agent), it means that the virus infection is interfered or blocked, and the test agent is deemed a candidate inhibiting infection of the virus in the cells.

Therefore, in a further aspect, the present invention provides a method of evaluating if a test agent inhibits virus infection, comprising:

(a) conducting a first detection as a control, which comprises

(i) providing a microcantilever detection device, which comprises a microcantilever comprising a contact area having a macromolecular material with host cells attached thereto, wherein the macromolecular material is hydrophilic and biocompatible;

(ii) loading a sample containing a virus that is capable of infecting the host cells to the contact area to allow the virus to contact and infect the host cells, and washing the contact area to remove free virus that does not infect the host cells, whereby the microcantilever causes a first deflection level, which continuously increases as the virus replicates in the host cells over time; and

(iii) measuring the first deflection level in a time course manner during a period of time so as to give a first deflection curve, which has a first slope representing the increase of the first deflection level caused by replication of the virus in the host cells,

(b) conducting a second detection in the same manner as in the first detection, except that the test agent is added to the sample to be loaded to the contact area, so as to give a second deflection curve, which has a second slope corresponding to the first slope in the first deflection curve;
(c) comparing the second slope in the second deflection curve with the first slope in the first deflection curve, wherein the second slope less steeper than the first slope indicates that the test agent is a candidate inhibiting infection of the virus in the cells.

As used herein, the term “test agent” is any molecule or substance to be evaluated for its effectiveness in inhibiting virus infection, such as a compound, an antibody, a natural product or extract, or a vaccine.

According to the invention, the first slop of the first deflection curve can represent the replication phase of the virus infection process in the cells. If the second slope of the second deflection curve is less steeper than the first slope of the first deflection curve (the control curve), it means that the increase of the deflection level is prevented by the test agent, indicating that the test agent interferes the virus infection in the cells, specifically inhibits entry and/or replication of the virus in the cells. As used herein, the second slope is less steep than the first slope, which may mean a relatively small increase in the deflection level in the same time interval. More specifically, the second slope (in absolute value) is about 50% or less, 40% or less, 30% or less, 20% or less, 10% or less, of the first slope (in absolute value).

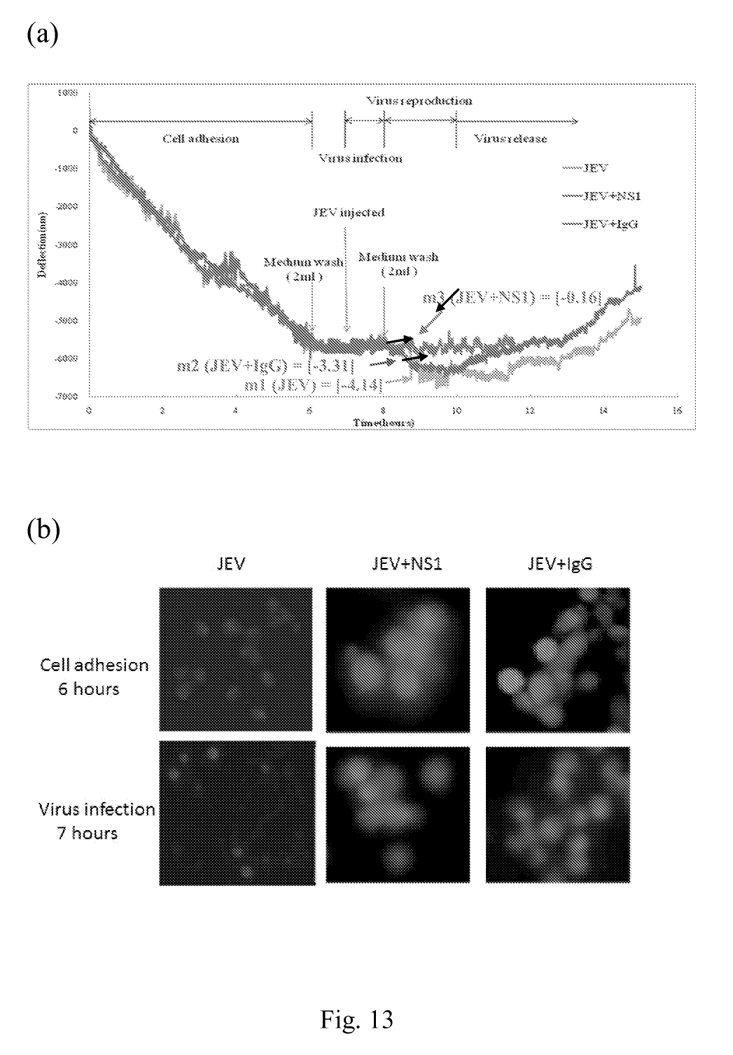

See Examples 4.1 and FIG. 13(a), for example. The first slope, m1 (JEV) is −4.14, representing the virus replication in the cells, and the second slope, m2 (JEV+IgG) −3.31, similarly steep to m1, showing that IgG cannot block the virus infection/replication, and in the contrast, m2 (JEV+NS1)−0.16, less steeper than the first slope, indicating that the agent NS1 blocks the virus infection/replication.

In addition, in principle, the first deflection curve represents a normal infection process of the virus in the cells, which typically shows a “turning point” where the deflection level achieves a maximum value, after which the deflection level stops increasing and starts to move back to the original level prior to loading the virus, which means that the virus and completes replication in the host cells and starts to leave the host cells (entering the transmission phase), and hence around the turning point, the defection curve typically forms an “V-like” or “L-like” profile (bouncing back), which is an important marker representing that the replication phase is completed and the virus is leaving the cells. See FIG. 12, at time point (e), for example. Accordingly, in one embodiment of the invention, one may also determine the effectiveness of the test agent in inhibiting virus infection, based on the presence or absence of such “turning point” or “V-like” or “L-like” profile in the second detection curve, wherein if the second detection curve does not show such “turning point” or “V-like” or “L-like” profile, the test agent is deemed a candidate inhibiting infection of the virus in the cells.

The various embodiments of the present invention are described in details below. The technical characteristics of the present invention will be more clearly presented by the following detailed descriptions about the various embodiments and claims.

### EXAMPLE

### Example 1

### Design and Fabrication of the Microcantilever Detection Device of the Invention

FIG. 2 (b) shows the fabrication procedure of a microcantilever biochip consisting of a silicon nitride microcantilever and PDMS microfluidics. Bulk micromachined microcantilevers were fabricated using commercial four-inch silicon wafers, which were first cleaned using standard Piranha solution and RCA cleaning processes. The fabrication procedures of microcantilevers began with a 1 μM low stress silicon nitride thin layer being deposited on a silicon wafer followed by photolithographic processes using AZ4620 photoresist and photomask-1, which defined the pattern of the π-shape microcantilevers by aligner (AB-M InC., U.S.A.). The whole area of the π-shaped microcantilever was 1000 nm in length and 1000 nm in width. The length and width of the front-bar of the π-shaped microcantilevers were 1000 μm and 200 μm, respectively. The PHEMA hydrogel microstructure for laden cells must be deposited on the center of the front-bar to increase the sensitivity of the microcantilever. The laser spot light used to measure the microcantilever deflections could be located on the sides of the front-bar to avoid damaging cells adhering to the PHEMA hydrogel. Moreover, the reflection location of the laser beam will be uncertain during the period of microcantilever deflection if the laser spot light is guided onto the 3D PHEMA microstructure and cells. The silicon nitride thin film was used as an anisotropic masking layer, and reactive ion etching (Crie-100, Advanced System Technology, Taiwan) was then followed to selectively etch away the masking layer using SF6 and O₂ gases. The uncovered portion of the silicon substrate was etched by potassium hydroxide solution to release the microcantilevers. These microcantilevers were then rinsed in acetone followed by methanol, and deionized water (DI water) as shown in FIG. 2 (b).

The fabrication procedures of PDMS microfluidics began with a silicon wafer coated with SU-8 photoresist to create a mold master, including an inlet, an outlet, several bubble traps, and a chamber for cells adhered and cultured on microcantilevers as shown in FIG. 2 (b). Following soft baking of SU-8 photoresist, the photomask-2 patterns were transferred to the SU-8 coated silicon wafer by the above-mentioned aligner. Post-exposure bake, development, and hard bake of the exposed SU-8 patterns were followed by pouring the mixture of PDMS onto the patterns. The prepolymer was mixed with its curing agents in a 10:1 ratio, and was degassed in a vacuum chamber by an aspirator (AS-3, Newlab Instruments, UK) for at least 30 min at room temperature before pouring the mixture onto the patterns. The master was then replicated with PDMS for several minutes at 90° C. as shown in FIG. 2 (b). However, the surface of the PDMS replica is hydrophobic; thus, its surface must be changed to have a hydrophilic property before coating with gelatin and bonding onto a silicon substrate. In this work, the PDMS replica was rinsed in ethanol and then subjected to oxygen plasma using the above-mentioned reactive ion etching apparatus Crie-100 for 50 sec at a power of 20 watts and an oxygen flow rate of 50 sccm. A thin gelatin film used as a sacrificial layer was then deposited onto the bottom of the cell laden chamber of the PDMS replica to create a space for cells to be laden and cultured and to avoid the hydrogel microstructure sticking to the PDMS replica when hydrogel was exposed to UV light from the top side of the PDMS replica as shown in FIG. 2(b). Finally, the surface modified PDMS cast was bonded onto the micromachined silicon wafer containing microcantilevers as shown in FIG. 2(b).

Photolithography was utilized to micropattern PHEMA onto microcantilevers. After injecting the PHEMA hydrogel solution into the inlet of the microcantilever-based biochip, a photomask-3 was used to selectively expose the PHEMA solution by UV light with an intensity of 10 mW/cm² for 120 seconds. Afterward, 40° C. deionized water was injected into the biochip to remove the uncrosslinked PHEMA solution and the gelatin layer, releasing the microcantilever. The biochips containing microcantilevers with PHEMA microstructures were then injected with DI water and exposed to UV light several times to completely remove the residual DMSO solvent in PHEMA microstructures and disinfect the whole chip. The size of the hydrogel microstructure on the π-shaped microcantilever was about 200 nm×200 nm.

### Example 2

### Design and Establishment of the Optical Detection System

An optical detection system having the microcantilever detection device as described was designed and established for monitoring and recording a viral infection process in host cells.

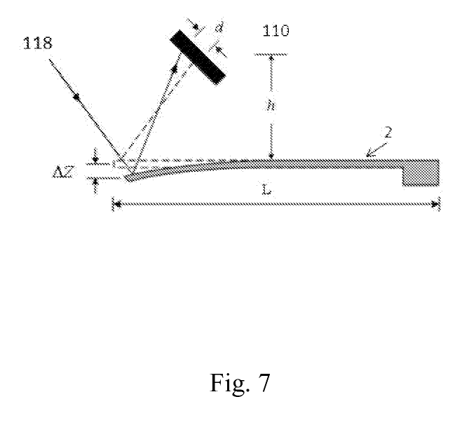

A low-power He—Ne laser was used as light source. The intensity of the laser light was equalized and the luminous flux was decreased via space filter and pinhole. The route of the light beam with defined size was refracted by a refractive lens, and then the light beam focused on the optical detection area of the microcantilever, and the beam reflected to the four-quadrant position sensing detector (Position-Sensitive detector, PSD) because of the optical lever principle. The displacement d of the reflected light was measured by the four-quadrant position sensing detector, from which the deflection of the microcantilever detection device was calculated, as shown in FIG. 7.

The deflection ΔZ of the microcantilever detection device may be calculated based on the optical lever principle and triangle geometry:

$$Δ Z = \frac{d}{h} L$$

d: the displacement of the reflected laser beam on the four-quadrant position sensing detector

h: the distance between microcantilever detection device and the four-quadrant position sensing detector

L: the length of the microcantilever detection device

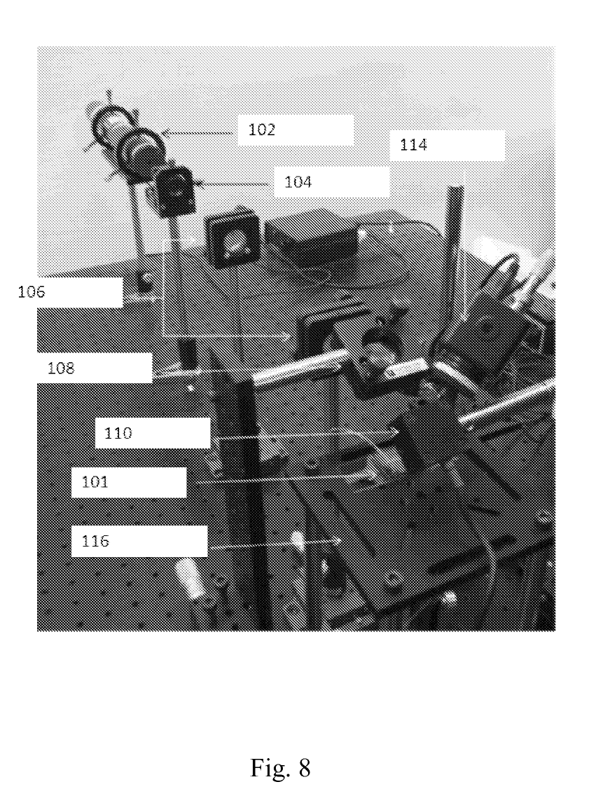

In the optical detection system of the present invention, a charge-coupled device (CCD) was also used to observe whether the laser beam was focused accurately on the optical detection area. The signal received by the four-quadrant position sensing detector was amplified by an amplifier and recorded in a computer. In addition, in the optical detection system of the present invention, fluorescence microscope CCD lens may also be used to observe synchronously the growing of cells on the hydrogel material, in order to further understand the complete viral infection process and compare it with the result of traditional viral infection detection methods. The optical detection system of the present invention is shown in FIG. 8.

### Example 3

### Monitoring and Recording a Viral Infection Process in Host Cells

3.1 Cell Preparation

BHK-21 cells (Baby hamster kidney cell line, ATCC CCL-10) were used in this example. Cells were cultured in the medium containing trypsin and ethylenediaminetetraacetic acid (EDTA) at 37° C., so that cells could adhere to the culture plate and grow.

3.2 Loading Cell Medium and Detecting the Deflection of the Microcantilever

1 ml medium containing the BHK-21 cells (1.6×10⁶ cells/ml) was injected into the microcantilever-based biochip at 100 μl/min flow rate by a syringe pump (Harvard Apparatus, USA), and then the pump was turned off. The volume of the chamber was around 290 μl. The chamber was in static condition after injecting to allow the BHK-21 cells to naturally adhere onto the hydrogel microstructure. A living and dead assay were then performed with ethidium homodimer and calcein AM. These two stains were added to PBS, and then injected into the chamber of the microcantilever based biochip in order to stain the BHK-21 cells laden on the PHEMA hydrogel microstructure. After rinsing with PBS, the cells were evaluated by a fluorescence microscopy (BX51, Olympus, Japan). According to the cell fluorescent signals, it was found that cells successfully adhered to the hydrogel material surface and most of them are healthily alive (data not shown).

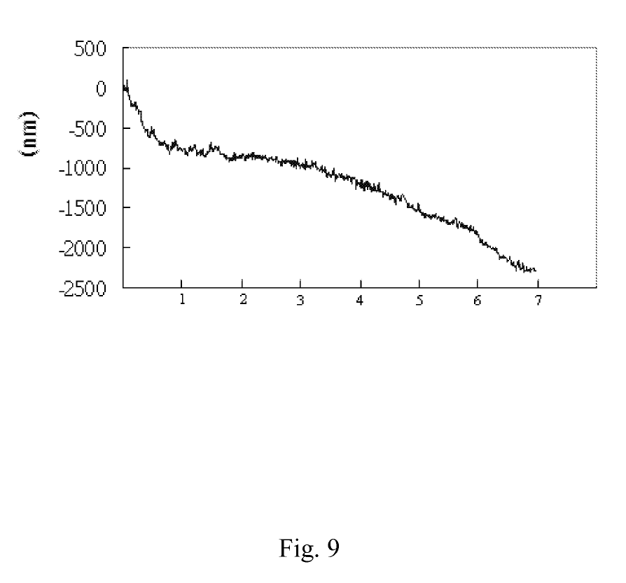

On the other hand, the deflection of the microcantilever were measured by the optical detection system as shown in FIG. 8, including a He—Ne laser source 102, a space filter 104, focus lens 106, refractive lens 108, a position sensing detector 110, a microcantilever detection device (chip) 101, a charge-coupled device (CCD) 114, and a sample stage 116. As shown in FIG. 9, the optical detection system of the present invention successfully detected the signals in responsive to the deflection generated by the microcantilever, wherein the deflection level continually increased during the cell-laden period.

3.3 Loading a Virus Sample and Monitoring and Recording the Infection process in host cells

After the host cells were attached to the hydrogel microstructure (7 hours), a phosphate buffer containing Japanese encephalitis virus was loaded into the microfluidic system whereby the virus contacted and entered the cells attached to the hydrogel material of the microcantilever detection device. The M.O.I (multiplicity of infection) value in this study was 1, namely one virus infecting one cell. It is known that the infection cycle of Japanese encephalitis virus including entering to the cells, replicating in the cells and leaving the cells was about 6 hour. Therefore, in this study, signals responsive to the deflection of the microcantilever detection device were collected and recorded for a 7-hour period of time.

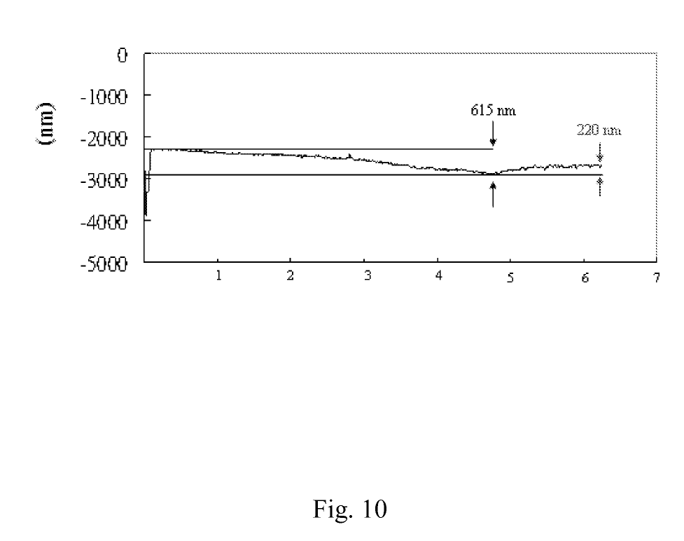

As shown in FIG. 10, after the phosphate buffer containing Japanese encephalitis virus was loaded, the microcantilever started to deflect downwards, which implied that the virus has entered the cells and started to replicate in the cells. The deflection level of the microcantilever continuously increased to a maximum value, 615 nm, at 4 hours and 40 minutes, after which, the deflection level stop increasing and exhibited a trend to turn back toward the level prior to loading the virus sample, which implied that the virus has completed the replication and started to leave the cells.

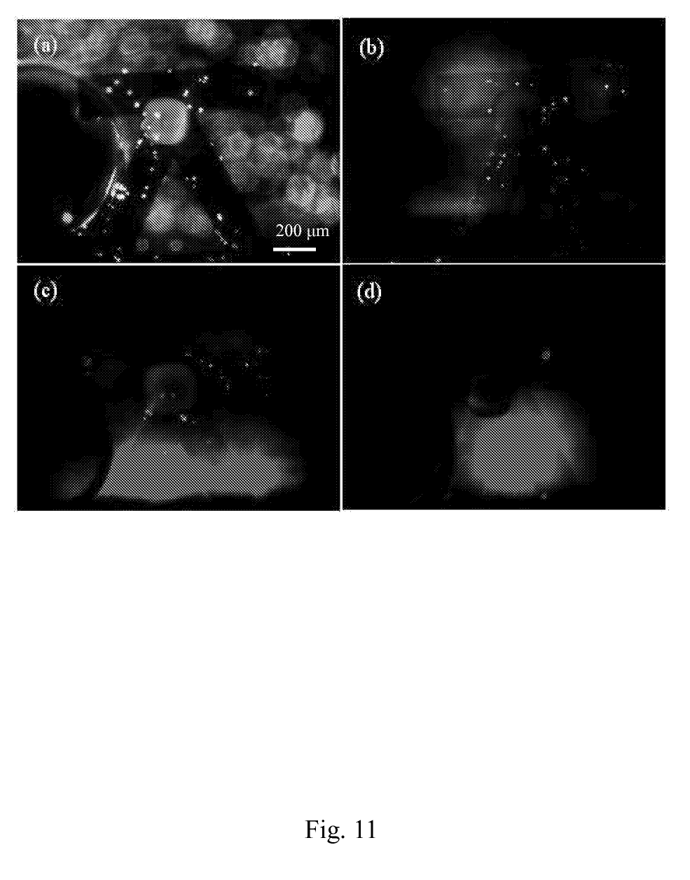

Simultaneous observations of the cells were conducted with a fluorescence microscope to confirm each phase of the viral infection process as described above. As shown in FIG. 11, in the control group (a), the BHK-21 cells loaded to the hydrogel material (11 hours), without loading Japanese encephalitis virus, were mostly healthily alive. In the experimental groups, the BHK-21 cells were attached to the hydrogel material (7 hours) and then the virus was loaded to the cells. Observation was made at 4 hours (b) and 7 hours (c) after loading the virus. It was found that the cells gradually died because of the viral infection and detached from the surface of the hydrogel material, which was consistent with the result of the decrease of the deflection level of the microcantilever. Subsequently, the microfluidic system was washed with phosphate butter, and it was observed that most of the cells died and detached from the surface of the hydrogel material (d).

A separate detection was conducted using BHK-21 cells (2.4×10⁶ cells/ml) and Japanese encephalitis virus (M.O.I: 1), within a period of 16 hours and 15 minutes, to monitor the viral infection process in the host cells and obtain a deflection curve according to the present invention. FIG. 12 shows the results.

Briefly, as shown in FIG. 12, at the time point (a) being 09:55:31 AM, the cells were loaded to the hydrogel material. After the cells were attached to the hydrogel material, the deflection curve showed the slope m1 (−4.96) during the time period from (a) to (b), about 6 hours and 38 minutes. At the time point (b) being 04:33:55 PM, 5 ml culture medium was added to wash away free cells that were not attached to the hydrogel material, whereby we could make sure that the following deflection was resulted from the subsequent viral infection, instead of the unwanted suspending cells. When the system went stable, the defection curve showed the slope m2 (−2.16) during the time period from (b) to (c). At the time point (c) being 07:40:36 PM, a sample containing the virus was loaded to the hydrogel material and then the deflection curve showed the slope m3 (−5.31) during the time period from (c) to (d). At the time point (d) being 08:37:48 PM, 5 ml culture medium was used to wash away the virus that did not enter the cells, whereby we could make sure that the following deflection was resulted from the subsequent viral replication, instead of the unwanted suspending virus. When the system went stable, the deflection curve showed the slope m4 (−1.42) during the period of time (d) to (e), which indicates that the virus was entering the cells and replicating in the cells, leading to increasing of the deflection level. Finally, at the time point (e) being 01:16:14AM, the deflection level reached the maximal value, after which the deflection level stop increasing and started to go towards to the value prior to loading of the sample, indicating that the virus completed replication in the host cells and was leaving the host cells (transmission phase).

### Example 4

### Determining the Potential Efficacy of a Test Agent to Inhibit Virus Infection

4.1 Japanese Encephalitis Virus (JEV)

BHK-21 cells were cultured as described in Example 3.1. The cells were loaded to the hydrogel microstructure and the deflection of the microcantilever were monitored and recorded as described in Example 3.2.

The cells (2.4×10⁶ cells/ml) were attached to the hydrogel microstructure for six hours and the unattached cells were removed by the medium to ensure that the following deflection of microcantilever was resulted from the subsequent virus infection, instead of unwanted suspending cells. One hour later, a sample containing JEV (M.O.I=1) was loaded into the hydrogel microstructure and after another one hour, the free virus was removed by the medium to make sure that the following deflection was resulted from the subsequent viral replication in the cells, instead of the unwanted free virus that did not enter the cells. FIG. 13 (a) shows the deflection curve as obtained (the JEV curve, green) which represents the infection process of JEV in the BHK-21 cells.

As shown in FIG. 13 (a), at eight hours, the microcantilever started to deflect downwards, which implied that the virus was continuously infecting the cells and started to replicate in the cells (the green curve). The deflection level continuously increased for about 2-3 hours (the replication phase). Later, the deflection level reached a maximum value, after which the deflection level stop increasing and started to go towards to the value prior to loading of the virus sample (move upwards), indicating that the virus has completed the replication and started to leave the cells.

On the other hand, a test sample containing (i) JEV and a non-specific antibody IgG (Goat anti Mouse IgG-HRP (Santa Cruz Biotechnolog, 200 μg/0.5 ml, 5000× dilution) or (ii) JEV and a specific antibody NS1 (a Monoclonal antibody E3 against the E protein of JEV, 5000× dilution, produced as ascites in Balb/c mice by injection of the producing hybridoma and purified by protein A chromatography, described in Wu S C, Lian W C, Hsu L C, Liau M Y. Japanese encephalitis virus antigenic variants with characteristic differences in neutralization resistance and mouse virulence. Virus Res 1997; 51:173-81) were independently loaded to the hydrogel microstructure and the resultant deflection curves were recorded, respectively. FIG. 13 (a) shows the results. See the JEV plus IgG curve (red) and the JEV plus NS1 curve (blue).

As shown in FIG. 13 (a), the JEV plus IgG curve (red, m2=−3.31) shows a similar profile to that of the JEV curve (green, m2=−4.14), indicating that the non-specific IgG cannot inhibit the virus infection, more particularly cannot inhibit the virus to enter the cells and/or replicate in the cells. On the contrary, the JEV plus NS1 curve (blue, m3=−0.16) shows a relatively less steeper slope (namely a relatively more steady slope) during the corresponding replication phase, indicating that the specific antibody NS1 inhibited the viral infection, more particularly inhibited the virus to enter the cells and/or replicate in the cells.

Fluorescence microscopic observation was conducted to confirm the results. FIG. 13 (b) shows the fluorescence microscopic images of the cells in the study. As shown in FIG. 13 (b), BHK-21 cells, after being attached to the hydrogel material (at six hours), were mostly healthily alive; the cells were then gradually died after loading JEV or JEV plus the non-specific IgG (seven hours after virus infection), but were still alive after loading JEV plus the specific antibody NS1 (seven hours after virus infection). The fluorescence microscopic observation is consistent with the deflection curve as recorded in FIG. 13 (a), demonstrating that the system of the invention is stable and reproducible for determining if a test agent can inhibit virus infection.

4.2 Dengue Virus (DV)

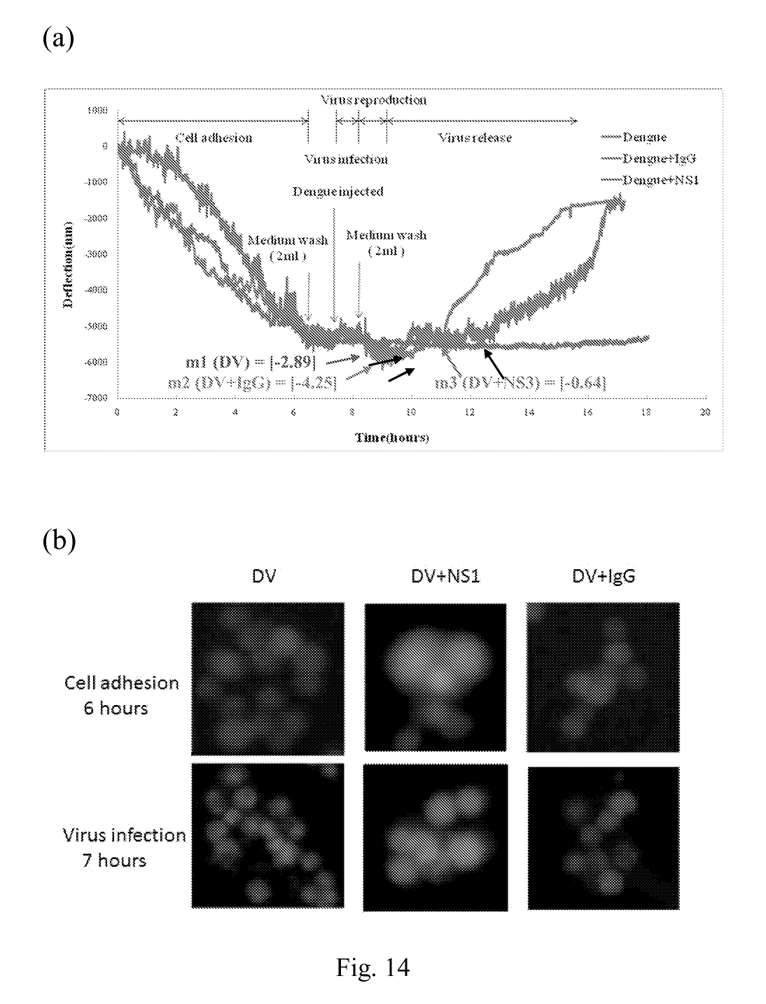

The system of the invention was also used to monitor and record the infection process of dengue virus (DV) in host cells. The experiment was performed as described in 4.1 (the cell concentration is 2.4×10⁶ cells/ml; M.O.I=1). FIG. 14 (a) shows the deflection curve as detected and recorded (the DV curve, purple) which represents the infection process of DV in the BHK-21 cells.

As shown in FIG. 14 (a), the replication phase occurred at 8-9 hours, lasting for about 1 hour, during which the deflection level continuously increased (move downwards). Subsequently, the deflection level stop increasing and started to go towards to the value prior to loading of the virus sample (move upwards), indicating that the replication phase was completed and the virus started to leave the cells and the cells gradually died.

A test sample containing (i) DV and a non-specific antibody IgG (Goat anti Mouse IgG-HRP (Santa Cruz Biotechnolog, 200 μg/0.5 ml, 5000× dilution)) or (ii) DV and a specific antibody NS3 (Rabbit anti DV2-NS3 (GeneTex, 1 mg/ml, 5000× dilution)) were independently loaded to the hydrogel microstructure and the resultant deflection curves were recorded, respectively. FIG. 14 (a) shows the results. See the DV plus IgG curve (blue) and the DV plus NS3 curve (red).

As shown in FIG. 14 (a), the DV plus IgG curve (blue, m2=−4.25) shows a similar profile to that of the DV curve (purple, m1=−2.89), indicating that the non-specific IgG cannot inhibit the virus infection, more particularly cannot inhibit the virus to enter the cells and/or replicate in the cells. On the contrary, the DV plus NS3 curve (red, m3=−0.64) shows a relatively less steeper slope (namely a relatively more steady slope), without continuously increasing of the deflection level to a maximum value or subsequently moving upward, indicating that the specific antibody NS1 inhibited the viral infection, more particularly inhibited the virus to enter the cells and/or replicate in the cells.

Fluorescence microscopic observation was conducted to confirm the results. FIG. 14 (b) shows the fluorescence microscopic images of the cells in the study. As shown in FIG. 14 (b), BHK-21 cells, after being attached to the hydrogel material (at six hours), were mostly healthily alive, whereas the cells were then gradually died after loading DV or DV plus the non-specific IgG (seven hours after virus infection). In the contrast, the cells were still alive after loading DV plus the specific antibody NS3 (seven hours after virus infection). The fluorescence microscopic observation is consistent with the deflection curve as recorded in FIG. 14 (a), demonstrating that the system of the invention is stable and reproducible for screening for a potential agent that can inhibit virus infection, especially to inhibit the virus to enter the cells and/or replicate in the cells.

The above results show that, the system and method of the present invention can be used for monitoring and recording viral infection process in host cells, as well as for screening for an agent that inhibits virus infection. The present invention provides a new platform technology to monitor and record viral infection process and to conduct a preliminary screening for a candidate agent for inhibiting virus infection, which is fast, simple, non-expensive and effective.

It is believed that a person of ordinary knowledge in the art where the present invention belongs can utilize the present invention to its broadest scope based on the descriptions herein with no need of further illustration. Therefore, the following descriptions should be understood as of demonstrative purpose instead of limitative in any way to the scope of the present invention. The publications referred to herein are incorporated by reference in their entireties.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for monitoring and recording a viral infection process, comprising:
(a) providing a microcantilever detection device, which comprises a microcantilever comprising a contact area having a macromolecular material attached thereon; wherein the macromolecular material is hydrophilic and biocompatible;
(b) loading host cells to the contact area to allow the host cells to be attached to the macromolecular material, and washing the contact area to remove unattached cells;
(c) loading a sample containing a virus capable of infecting the host cells to the contact area to allow the virus to contact and infect the host cells attached thereto, and washing the contact area to remove free virus that does not infect the host cells, whereby the microcantilever causes a deflection level;
(d) measuring the deflection level of the microcantilever in a time course manner during a period of time so as to give a deflection curve; and
(e) monitoring and recording an infection process of the virus in the host cells based on the deflection curve, in which when a continued increase of the deflection level appears, it indicates that the virus replicates in the host cells, and when the deflection level achieves a maximum value, it indicates that the virus completes replication in the host cells and starts to leave the host cells.

2. The method of claim 1, wherein the macromolecular material is a hydrogel material.

3. The method of claim 2, wherein the hydrogel material is selected from the group consisting of polyhydroxyethylmethacrylate (PHEMA) hydrogel, polyethylene glycol diacrylate (PEGDA) hydrogel, gelatin methacrylate (GelMA) hydrogel, alginate hydrogel, alginate hydrogel, chitosan hydrogel and agarose hydrogel.

4. The method of claim 1, wherein the microcantilever is π-shaped.

5. The method of claim 1, wherein the deflection is measured by an optical detection approach, an acoustic detection approach, an electric detection approach, or a magnetic detection approach.

6. The method of claim 1, wherein the microcantilever detection device further comprises a microfluidic system, through which the sample containing the virus is loaded to the contact area of the microcantilever to allow the virus to contact and infect the cells attached thereto.

7. A method of evaluating if a test agent inhibits virus infection, comprising:
(a) conducting a first detection as a control, which comprises
(i) providing a microcantilever detection device, which comprises a microcantilever comprising a contact area having a macromolecular material with host cells attached thereto, wherein the macromolecular material is hydrophilic and biocompatible;
(ii) loading a sample containing a virus that is capable of infecting the host cells to the contact area to allow the virus to contact and infect the host cells, and washing the contact area to remove free virus that does not infect the host cells, whereby the microcantilever causes a first deflection level, which continuously increases as the virus replicates in the host cells over time; and
(iii) measuring the first deflection level in a time course manner during a period of time so as to give a first deflection curve, which has a first slope representing the increase of the first deflection level caused by replication of the virus in the host cells,
(b) conducting a second detection in the same manner as in the first detection, except that the test agent is added to the sample that is to be loaded to the contact area, so as to give a second deflection curve, which has a second slope corresponding to the first slope in the first deflection curve;
(c) comparing the second slope in the second deflection curve with the first slope in the first deflection curve, wherein the second slope less steeper than the first slope indicates that the test agent is a candidate inhibiting infection of the virus in the cells.

8. The method of claim 7, wherein the macromolecular material and the second macromolecular material are a hydrogel material.

9. The method of claim 8, wherein the hydrogel material is selected from the group consisting of polyhydroxyethylmethacrylate (PHEMA) hydrogel, polyethylene glycol diacrylate (PEGDA) hydrogel, gelatin methacrylate (GelMA) hydrogel, alginate hydrogel, alginate hydrogel, chitosan hydrogel and agarose hydrogel.

10. The method of claim 7, wherein the microcantilever are π-shaped, H-shaped, K-shaped, Y-shaped, X-shaped, T-shaped, W-shaped or M-shaped.

11. The method of claim 7, wherein the first deflection and the second deflection are measured by an optical detection approach, an acoustic detection approach, an electric detection approach, or a magnetic detection approach.

12. A system for monitoring and recording an infection process of a virus in host cells, comprising:
(a) a microcantilever detection device, which comprises a microcantilever comprising a contact area having a hydrophilic and biocompatible macromolecular material attached thereon for fixing the host cells, and a signal detecting area; wherein when the host cells are attached to the contact area, and the virus is then loaded to the contact area and contacts and infects the host cells, the microcantilever produces a deflection level that is detectable through the signal detecting area;
(b) a signal detecting device, comprising a signal producing means for producing a detectable signal responsible to the deflection and a signal receiving means for receiving the detectable signal and converting it to an outputting signal; and
(c) a signal processing device for receiving the outputting signal and converting it to a data so as to give a deflection curve in a period of time of measurement, which presents the infection process of the virus in the host cells.

13. The system of claim 12, wherein the signal detecting device is established based on an optical detecting approach, an acoustic detecting approach, an electric detecting approach, or a magnetic detecting approach.

14. The system of claim 12, wherein the signal detecting device is establish based on an optical detecting approach, which comprises a laser source, a spatial filter, a focusing lens set, refractive lens, a position sensing detector, wherein the laser source provides a beam of laser light that goes through the spatial filter to form an uniform beam, which then goes through the focusing lens set to form a parallel beam, which further goes through the refractive lens to form a first reflected beam, which subsequently focuses on the signal detecting area of the microcantilever detection device and forms a second reflected beam, and the position sensing detector receives the second reflected beam and converts it to an electrical outputting signal.

15. The system of claim 12, wherein the system further comprises a charge coupled device for observing whether the first reflected beam focuses on the signal detecting area of said microcantilever detection device.
